SEQUENCE LISTING (43 sequences)

SEQ ID NO: 1 - protein, 40 aa, Hepatitis C virus
VPAAYAAQGYKVLVLNPSVAATLGFGAYMSKAHGIDPNIR

SEQ ID NO: 2 - protein, 15 aa, Artificial Sequence
  C-amidated peptide used for the production of hybridoma B-3
GYKVLVLNPSVAATC

SEQ ID NO: 3 - protein, 11 aa, Artificial Sequence
  peptide inside the IgG-Fc region
EPQVYTLPPSR

SEQ ID NO: 4 - protein, 11 aa, Artificial Sequence
  conserved IgG-Fc epitope from 345 to 355
EPQVYTLPPSR

SEQ ID NO: 5 - protein, 11 aa, Artificial Sequence
  mutated IgG-Fc epitope from 345 to 355
EPQVYTLPPSQ

SEQ ID NO: 6 - protein, 20 aa, Artificial Sequence
  peptide used for the production of hybridoma B3-18
VLVLNPSVAATLGFGAYMSK

SEQ ID NO: 7 - DNA, 311 bp, Homo sapiens
acccagtctccaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctccaacgttcggagggggcaccaagctggaaatcaaacg

SEQ ID NO: 8 - protein, 103 aa, Homo sapiens
TQSPKFMSTSVGDRVSVTCKASQNVGTNVAWYQQKPGQSPKALIYSASYRYSGVPDRFTGSGSGTDFTLTISNVQSEDLAEYFCQQYNSYPPTFGGGTKLEIK

SEQ ID NO: 9 - DNA, 336 bp, Homo sapiens
  misc_feature (306)..(306): n is a, c, g, or t
atccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaagggacggtttgccttctctttggaaacctctgccagcactgcctatttgcagatcaacaacctcaaaaatgaggacacggctacatatttctgtgcaagattgaagcggtatttntatgctatggactactggggtcaaggaacc

SEQ ID NO: 10 - protein, 112 aa, Homo sapiens
  misc_feature (102)..(102): Xaa can be any naturally occurring amino acid
IQLVQSGPELKKPGETVKISCKASGYTFTNYGMNWVKQAPGKGLKWMGWINTNTGEPTYAEEFKGRFAFSLETSASTAYLQINNLKNEDTATYFCARLKRYXYAMDYWGQGT

SEQ ID NO: 11 - protein, 15 aa, Artificial Sequence
  Germ line heavy chain V1-2/D2-15/J4
ASGYTFTGYYMHWVR

SEQ ID NO: 12 - protein, 15 aa, Artificial Sequence
  24-38 patient heavy chain V1-2/D2-15/J4
ASGYTFTDYYIHWVR

SEQ ID NO: 13 - protein, 18 aa, Artificial Sequence
  Germ line heavy chain V1-2/D2-15/J4
VTMTRDTSISTVYMELSR

SEQ ID NO: 14 - protein, 18 aa, Artificial Sequence
  68-85 patient heavy chain V1-2/D2-15/J4
VTVTRDTSINTVYMELSR

SEQ ID NO: 15 - protein, 21 aa, Artificial Sequence
  Germ line light chain V3-15/J1
ASQSVSSNLAWYQQKPGQAPR

SEQ ID NO: 16 - protein, 21 aa, Artificial Sequence
  25-54 patient light chain V3-15/J1
ASQSVSSNLAWYQQQPGQAPR

SEQ ID NO: 17 - protein, 15 aa, Artificial Sequence
  Germ line heavy chain V1-69/D3-22/J4
ASGGTFSSYAISWVR

SEQ ID NO: 18 - protein, 15 aa, Artificial Sequence
  43-57 patient heavy chain V1-69/D3-22/J4
ASGGTFSSYGISWVR

SEQ ID NO: 19 - protein, 13 aa, Artificial Sequence
  Germ line heavy chain V1-69/D3-22/J4
STSTAYMELSSLR

SEQ ID NO: 20 - protein, 13 aa, Artificial Sequence
  94-106 patient heavy chain V1-69/D3-22/J4
STSTVYMELTSLR

SEQ ID NO: 21 - protein, 9 aa, Artificial Sequence
  Germ line light chain V3-20/J1
LLIYGASSR

SEQ ID NO: 22 - protein, 9 aa, Artificial Sequence
  29-37 patient light chain V3-20/J1
LLVYGASNR

SEQ ID NO: 23 - protein, 9 aa, Artificial Sequence
  Germ line light chain V3-20/J1
ATGIPDRFS

SEQ ID NO: 24 - protein, 9 aa, Artificial Sequence
  38-46 patient light chain V3-20/J1
ATGIPDRFR

SEQ ID NO: 25 - protein, 14 aa, Artificial Sequence
  Germ line light chain V3-20/J1
GSGSGTDFTLTISR

SEQ ID NO: 26 - protein, 14 aa, Artificial Sequence
  47-60 patient light chain V3-20/J1
GSGSGTDFTLTITR

SEQ ID NO: 27 - protein, 19 aa, Artificial Sequence
  Germ line light chain V3-20/J1
LEPEDFAVYYCQQYGSSPR

SEQ ID NO: 28 - protein, 19 aa, Artificial Sequence
  61-79 patient light chain V3-20/J1
LEPEDFALYYCQQYGSSPR

SEQ ID NO: 29 - protein, 21 aa, Artificial Sequence
  Germ line light chain V3-20/J1
SQSVSSSYLAWYQQKPGQAPR

SEQ ID NO: 30 - protein, 22 aa, Artificial Sequence
  7-28 patient light chain V3-20/J1
TSQSVSSTYLAWYQQTPGQAPR

SEQ ID NO: 31 - protein, 14 aa, Artificial Sequence
  Germ line heavy chain V3-7/D3-22/J4
QDGESEKYYVDSVK

SEQ ID NO: 32 - protein, 14 aa, Artificial Sequence
  61-75 patient heavy chain V3-7/D3-22/J4
EDGESEKYYVDSVK

SEQ ID NO: 33 - protein, 11 aa, Artificial Sequence
  Germ line heavy chain V3-7/D3-22/J4
NSLYLQMNSLR

SEQ ID NO: 34 - protein, 11 aa, Artificial Sequence
  83-87 patient heavy chain V3-7/D3-22/J4
NSLDLQMSSLR

SEQ ID NO: 35 - protein, 21 aa, Artificial Sequence
  Germ line light chain V3-15/J1
ASQSVSSNLAWYQQKPGQAPR

SEQ ID NO: 36 - protein, 21 aa, Artificial Sequence
  25-46 patient light chain V3-15/J1
ASQSVSSNLAWYQQRPGQAPR

SEQ ID NO: 37 - protein, 9 aa, Artificial Sequence
  Germ line heavy chain VH4-59
VTISIDVSK

SEQ ID NO: 38 - protein, 9 aa, Artificial Sequence
  66-74 patient heavy chain VH4-59
VTISIDTSK

SEQ ID NO: 39 - protein, 20 aa, Artificial Sequence
  epitope from 1251 to 1270
VLVLNPSVAATLGFGAYMSK

SEQ ID NO: 40 - protein, 9 aa, Artificial Sequence
  epitope from 1271 to 1279
AHGINPNIR

SEQ ID NO: 41 - protein, 11 aa, Artificial Sequence
  epitope from 1238 to 1248
VPAAYAAQGYK

SEQ ID NO: 42 - protein, 15 aa, Artificial Sequence
  epitope from 1284 to 1298
TITTGSPITYSTYGK

SEQ ID NO: 43 - protein, 8 aa, Artificial Sequence
  epitope from 1379 to 1387
AIPLEVIK

(19) United States
(12) Patent Application Publication
(10) Pub. No.: US 2008/0248042 A1
(43) Pub. Date: Oct. 9, 2008

(54) Monoclonal antibody cross-reactive against infective agent causing a B-cell expansion and IgG-Fc

(75) Inventors: Valli De Re, Aviano (PN) (IT); Maria Paola Simula, Aviano (PN) (IT); Laura Caggiari, Aviano (PN) (IT); Annunziala Gloghini, Aviano (PN) (IT)

Correspondence Address: NIXON & VANDERHYE, PC, 901 NORTH GLEBE ROAD, 11TH FLOOR, ARLINGTON, VA, 22203, US

(73) Assignee: IRCCS CENTRO DI RIFERIMENTO ONCOLOGICO DI AVIANO,, Aviano (PN) (IT)

(21) Appl. No.: 11/783,297

(22) Filed: Apr. 5, 2007

(51) Int. Cl. A61K 39/395; A61P 31/12; A61P 35/00; A61P 37/00; C07K 16/10; C07K 16/18; C07K 16/34; C12N 5/12; C12N 5/22; C12N 7/01; G01N 33/564

(52) U.S. Cl. 424/139.1; 424/142.1; 435/235.1; 435/326; 435/331; 436/506; 530/387.1; 530/387.9; 530/388.1; 530/388.3

(57) ABSTRACT

This invention disclosed a monoclonal antibody or a derivative thereof which is cross-reactive against the immunogenic sequence of an infective agent causing a B-cell expansion and IgG-Fc, said infective agent is selected from the group consisting of staphylococcus, HCV, HSV-1, HSV-2, varicella-zoster, CMV, and EBV. The monoclonal antibody is cross-reactive against helicase domain of HCV-NS3 and human IgG-Fc, in particular is cross-reactive against NS3₁₂₄₆₋₁₂₅₈ and IgG-Fc₃₄₅₋₃₅₅. Hybridoma producing the antibody of is also provided. Methods for treating an infection, or for treating an autoimmune disease related to an infective agent, said agent causing B-cell expansion, type II mixed cryoglobulinemia, HCV-related neoplastic disease, non-Hodgkin lymphoma are disclosed. Also disclosed are methods for detecting an antigen responsible for inducing and maintaining B-cell activation and for selecting a patient suffering from an autoimmune or a neoplastic disease related to an infective agent, said agent causing B-cell expansion. Pharmaceutical compositions and immunoassays are also comprised in the invention.

The present invention relates to medicaments useful in the treatment of infectious, neoplastic and autoimmune diseases, in particular to infections from an infective agent causing a B-cell expansion and autoimmune and neoplastic related diseases.

More in particular, the present invention relates to monoclonal antibodies and to their use as medicaments, diagnostic tools and for drug design.

### BACKGROUND OF THE INVENTION

Abnormal B-cell activation, hypergammaglobulinemia, autoantibody production and immune complex formation are common features of chronic inflammatory conditions, including persisting viral infections. Although a viral role has not yet been established, chronic B-cell stimulation is of particular interest in hepatitis C virus (HCV) infection because it has been shown to be related to the increased risk of neoplastic transformation (Gisbert J P et al., Gastroenterology, 2003, 125:1723-1732; Mele A et al, Blood, 2003, 102, 996-999). Indeed, it is documented that B-cell non Hodgkin's lymphoma (NHL) can occur in persistently HCV-infected patients with a history of type II mixed cryoglobulinemia (MC), a chronic immune complex-mediated disease with underlying B-cell clonal proliferation (Dammacco F et al, Semin Liver Dis, 2000, 20, 143-157; De Re V et al, Blood, 2000, 96, 3578-3584; De Re V et al, Int J Cancer, 2000, 87, 211-216; Gasparotto D et al, Leuk Lymphoma, 2002, 43, 747-751).

Type II MC is serologically characterized by the presence of cold-precipitable protein complexes composed of mono/oligoclonal IgM with rheumatoid factor (RF) activity, bound to oligo- or polyclonal IgG (Brouet J C et al, Am J Med, 1974; 57, 775-788; De Re V et al, Rheumatology, 2006, 45(6), 685-93, 2006 Jan. 6, Epub). Morphologically it presents both bone marrow and intrahepatic multifocal B-cell lymphoid infiltrates (Sansonno D et al, Eur J Immunol, 2004, 34, 126-136). It is well documented that the vast majority of patients with symptomatic type II MC are infected with HCV and that more than 40% of the patients with chronic HCV infection have asymptomatic MC (Agnello V et al, N Engl J Med, 1992, 327, 1490-1495; Ferri C et al, Arthritis Rheum, 1991, 34, 1606-1610).

The mechanisms through which HCV infection leads to RF production, MC and NHL, as well as whether these conditions are related to the lack of antiviral function, are yet unknown. Current speculation is that HCV persistence contributes to oncogenesis by greatly expanding clones of immunoglobulin secreting B-cells which, depending on genetic and environmental factors, may undergo mutational events that cause the development of a malignant lymphoma. This is a similar mechanism to that hypothesized for Helicobacter pylori and mucosa associated lymphoid tissue (MALT) lymphomas (Hussell T et al, Lancet, 1993, 342, 571-574).

Previous studies have demonstrated that the BCR repertoire expressed by MC-II B-cell lymphoproliferations and HCV-associated NHL is not random, with V1-69/V3-20; V3-7/V3-15, V4-59/V3-20 variable heavy (VH)/variable kappa light (VL) chain gene combinations the most represented. The model of an antigen-driven origin for these lymphoproliferative disorders is based upon sequence data on BCR. Several studies demonstrated in fact the presence of IgV genes mutations compatible with a germinal center (GC) or post-GC derivation, a replacement/silent mutation ratio consistent with the maintenance of a functional structure of the BCR and the presence of intraclonal heterogeneity (De Re V et al, Eur J Immunol, 2002, 32, 903-910; Bende R J et al, J Exp Med, 2005, 201, 1229-1241). These notions are further strongly supported by the observation that a proportion of HCV-related MC and NHL are curable by virus eradication (Kelaidi C et al, Leukemia, 2004, 18, 1711-1716; Levine A M et al., N Engl J Med, 2003, 349, 2078-2079; Emens L A et al, N Engl J Med, 2002, 347, 2168-2170, Mazzaro C et al, N Engl J Med, 2002, 347, 2168-2170; Hermine O et al, N Engl J Med, 2002, 347(2), 89-94). Conceivably, the similarity in the structure of the variable BCR region, may account for selection of B-cells expressing specific and common reactivity.

Understanding the nature of the B-cell-stimulating antigen has proven extremely difficult. One reason relates to the difficulty in isolating expanded cells that are mostly confined to restricted areas of the bone marrow or liver. Another reason is related to the complexity of analyzing the BCR specificity of expanded cells, which are present at low concentrations among tissue-resident, nonspecific, B-cell populations and which also frequently present small differences among themselves because of the presence of an intraclonal diversity. Moreover, at DNA level, the bone marrow B-cell proliferation is oligoclonal in the majority of patients with type II MC. A further reason is the difficulties in obtaining reliable information from comparing the amino acid sequences of BCR and of antibodies of known specificity. Finally, although information could be gained from the cloning of the VH and VL genes into an immunoglobulin expression vector, the strategy is cumbersome and does not guarantee that a folded protein is produced as in vivo (Guo J Q et al, J Biotechnol, 2003, 102, 177-189).

Therefore, it is desirable to have available a research tool, in particular a protein, capable of mimicing as closest as possible the natural reactivity of a B cell during infection from an infective agent causing a B-cell expansion. This protein will also be useful in diagnosis and therapy.

All these problems make very difficult to provide an efficient drug for the treatment of this kind of diseases, in particular autoimmune and/or neoproliferative diseases, more in particular, diseases related to HCV infection.

Accordingly, there is a strongly felt need for a method of treating infection-related diseases, in particular autoimmune diseases, such as type II cryoglobulinemia, or neoplastic diseases, such as non-Hodgkin lymphoma, more in particular related to HCV infection.

In particular, it would be convenient to have a therapeutic and/or diagnostic tool targeted to HCV helicase, since this protein is a well-recognized target for fighting HCV infection (see US 2004/0253577A1 and related references).

It is well known that HCV infections are difficult to treat and the currently available clinical treatment, based on the combined use of interferon and ribavirin, is poorly effective (one patient of four) and expensive (Hoofnagle et al, 1994). Antibodies against NS3 are a proposed solution. US 2004/0214994 discloses a human recombinant antibody against NS3. This antibody is obtained with a sophisticated technique of genetic engineering, starting from the full length NS3 coding sequence obtained from the HCV genoma.

US2004/0142857A1 discloses a murine anti-idiotypic, monoclonal antibody and its use in diagnosis and therapy of HCV and HCV-related diseases, such as HCV-related B cell lymphoma. The antibody is obtained by immunization of mice with HIVIG.

WO 03/022296 discloses method and compositions for treating immune complex associated disorders, among which cryoglobulinemia is cited. This reference provides a method comprising administering to a subject affected by such a disorder a compound that either 1) inhibits formation of the immune complex either by preventing formation and/or binding to the TLR, or 2) interferes with binding of autoantigen-containing immune complex to the TLR, or 3) inhibits signalling pathways by dual engagement of BCR and TLR (in B cells) of FcR and TLR (in dendritic cells) via immune complexed or uncomplexed autoantigens.

However, the above cited references do not solve the problem of a direct relationship between HCV infection and cryoglobulinemia and HCV-related proliferative diseases, such as NHL.

### SUMMARY OF THE INVENTION

It has been discovered by the present inventors that the VH and VL chains amino acid sequences of the cryoprecipitable monoclonal IgM, match those deduced from the DNA analysis of the IgV genes of monoclonal B-cells isolated from patients with type II MC. This fact ensures that, in patients with an established monoclonal pattern, the IgM component of immune complexes represents the circulating counterpart of the BCR expressed on the surface of expanded B-cells and, therefore, can be exploited to identify the putative antigen involved in inducing and maintaining B-cell activation.

The present invention is based upon the above discovery and is embodied in different objects.

An object of the present invention is a monoclonal antibody or a derivative thereof which is cross-reactive against the immunogenic sequence of an infective agent causing a B-cell expansion, and IgG-Fc.

In particular, another object of the present invention is a monoclonal antibody or a derivative thereof, wherein said infective agent is selected from the group consisting of HCV, staphylococcus, HSV-1, HSV-2, varicella-zoster, CMV, and EBV.

In a preferred embodiment, it is an object of the present invention a monoclonal antibody and derivatives thereof which are cross-reactive against HCV-NS3 and IgG-Fc, said antibody being useful for detecting antigen(s) responsible for inducing and maintaining B-cell activation, in research, therapeutic and diagnostic applications.

Another object of the present invention are isolated, monoclonal IgM from a patient suffering from an infection from an infective agent causing a B-cell expansion, in particular suffering from type II mixed cryoglobulinemia, and their use as research tool, as well as diagnostic and therapeutic means.

Still another object of the present invention are immunodominant epitopes of the NS3₁₂₃₈₋₁₂₇₉ region of HCV genoma, in particular the peptide VPAAYAAQGYKVLVLNPSVAATLGFGAYMSKAHGIDPNIR, more in particular GYKVLVLNPSVAAT C(amide) used for the production of the hybridoma Called B-3); and the peptide EPQVYTLPPSR inside the Fc-IgG region. These peptides are epitopes, as defined in the following section of the detailed disclosure of the invention.

Another object of the present invention is a pharmaceutical composition comprising the monoclonal antibody or a derivative thereof or the isolated IgM or the above epitope peptides above mentioned or their combinations.

Methods of research for the etiopathogenic mechanisms sustaining the B cell proliferation above reported, diagnosis and treatment of infections and/or related diseases using the above objects are also enclosed within the scope of the present invention. In particular, infections from agent causing a B-cell expansion, such as infections from HCV, staphylococcus, HSV-1, HSV-2, varicella-zoster, CMV, and EBV and autoimmune or neoplastic related diseases are of interest.

Epitope of Fc-IgG are of interest in the present invention, since they can be used as delivery for drugs to expanding B cells.

These and other objects of the invention will be now disclosed in detail also by means of examples.

### DETAILED DISCLOSURE OF THE INVENTION

Although not limiting the scope of the present invention, the following definitions are offered for a better understanding of the invention.

“Monoclonal antibody” refers to an antibody composition having a homogeneous antibody population. This term is not limited by the manner by which the monoclonal antibody is obtained, nor the source from which it is obtained. Therefore, this term comprises monoclonal antibodies obtained from hybridomas from different animal species, for example murine hybridomas, as well as human monoclonal antibodies, see for example Cote et al, Monoclonal Antibodies and Cancer Therapy, Alan R Liss, 1985, and other similar publications.

“Monoclonal antibody derivative” refers to modifications of the antibody which do not substantially modify the intended or expected antibody activity according to the present invention. The antibody can be whole, a Fab, single chain, single domain heavy chain, etc. as provided in the art, see for example US 2005/0106142 and the references cited therein. The present invention relates also to antibody fragments or antibody chimera (such as, for example, a mouse-human chimera). The present invention provides an antibody or antibody fragment or antibody chimera or comprising at least one of a CDR of the variable light chain of the monoclonal antibody and/or a CDR of the variable heavy chain of the monoclonal antibody. The antibody or antibody fragment or antibody chimera or immunoglobulin molecule of the present invention may be an antibody, an Fv fragment, an Fab fragment, a F(ab)₂ fragment, a single chain antibody, or a multimeric antibody.

“Biological sample” refers to a sample of tissue or fluid isolated from a subject including, but not limited to, for example, blood, plasma, serum, fecal matter, urine, bone marrow, bile, spinal fluid, lymph fluid, samples of the skin, external secretions from the body, such as from skin, respiratory, intestinal and genitourinary tracts, tears, saliva, milk, blood cells, organs, biopsies and also samples in vitro cell culture constituents, for example conditioned media resulting from the growth of cells and tissues in culture medium, for example recombinant cells and cell components.

“Immune complex” means the combination formed by the interaction of an antibody and at least one epitope of one or more antigens.

“Epitope” means in the context of the present invention a sequence of at least 3 to 5, preferably about 5 to 10 or 15 and not more than 1,000 amino acids which define a sequence that by itself or as part of a larger sequence, binds to an antibody generated in response to such sequence. The present invention comprise not only sequences identical to the peptide above disclosed, but also modifications to the native sequence, such as deletions, additions and substitutions, generally conservative in nature, provided that the resulting modified peptide substantially maintains the reactive capability of forming the immune complex with the antibody of interest or, vice versa, the antibody of interest or a modification thereof substantially maintains the capability of recognizing the modified epitope and forming the immune complex.

“Immunodominant epitopes” means subunits of the antigenic determinant that are most easily recognized by the immune system and thus most influence the specificity of the induced antibody. There is no critical upper limit to the length of the fragment, which can comprise nearly the full length of the protein sequence, or even a fusion protein comprising two or more epitopes from the HCV proteoma.

According to the discovery on which the present invention is based, it has been found that the BCR of bone marrow monoclonal B-cells share the same antigen-binding fragment (Fab) with the cryoprecipitated IgM of the same patient. In some cases the same identity in patients displaying an oligoclonal B-cell pattern could be demonstrated.

According to one embodiment of the present invention, the isolated, monoclonal cryoprecipitable IgM from a patient with type II mixed cryoglobulinemia are the circulating counterpart of the BCR expressed on the surface of expanded B-cells. Said IgM are useful, at least in those cases where the BCR-IgM identity is demonstrated, as a tool to identify the putative antigen involved in inducing and maintaining B-cell proliferation.

Accordingly, a further object of the present invention is a method for the identification of the putative antigen involved in inducing and maintaining B-cell proliferation in a patient suffering from an autoimmune or neoplastice disease related to an infective agent causing a B-cell expansion. An example of such disease is type II mixed cryoglobulinemia.

This embodiment of the invention is carried out by means of diagnostic kit.

The invention will be now fully disclosed by reference to a preferred embodiment relating to HCV infection and HCV-related autoimmune and neoplastic disease.

### DESCRIPTION OF THE PREFERRED EMBODIMENT

Isolated, cryoprecipitated IgMs show a weak reactivity against the helicase domain of the NS3 protein in some subjects, whereas they show higher reactivity against Fc peptide in most of subjects. This makes possible providing a diagnostic kit comprising the Fc peptide for detecting those subjects who could be in need of a treatment for the B-cell proliferations in object.

It is known that during HCV infection NS3 can induce an important humoral and cellular immune response (Ou-Yang P et al, J Med Virol, 1999, 57, 345-350; Puoti M et al, Hepatology, 1992, 16, 877-881; Sallberg M et al, J Gen Virol 1996, 77, 2721-2728; Takaki A et al, C. Nat Med, 2000, 6, 578-582; Day C L et al, J Virol, 2002, 76, 12584-12595; Diepolder H M et al, Lancet, 1995, 346, 1006-1007). Nevertheless the structural details about the antigen presenting cells and the B cell epitope recognition in vivo, are still unknown. As a further problem solved by the present invention, applicants identified epitopes within the NS31238-1279 region by epitope excision approach. Interestingly, the same fragment NS31238-1279 is comprised in one of the two previously reported regions as immunodominant for B cells (AA 1250-1334; AA 1359-1449). Additionally, we found that all IgM present reactivity against human IgG, in agreement with our previous results from HCV-MC related NHL (De Re V, et al., Blood 2000; 96: 3578-3584). By epitope excision approach, a unique peptide, Fc₃₄₅₋₃₅₅ EPQVYTLPPSR, was identified as an epitope and reactivity was confirmed using this synthetic peptide in ELISA. The Fc₃₄₅₋₃₅₅ peptide is localized on the CH3 domain of IgG and it is part of the dimeric interface of the immunoglobulin. Its sequence is conserved among the different IgG classes except for IgG₄ which has a single mutation (EPQVYTLPPSQ). Although the high diversity of the NS3 and Fc epitopes seems to exclude molecular mimicry, overall data suggest that IgM antibodies against the NS3₁₂₃₈₋₁₂₇₉ domain, have cross-recognition for Fc₃₄₅₋₃₅₅.

In another embodiment, the present invention provides a monoclonal antibody endowed with this cross-reaction.

The monoclonal antibody is obtained by immunizing a mouse with the NS3₁₂₄₆₋₁₂₅₈ peptide, which was identified as an NS3 epitope in a patient affected by type II mixed cryoglobulinemia. The obtained hybridoma (hereinafter called B3-18, deposited with Centro di Biotecnologie Avanzate (CBA) Interlab Cell Line Collection (ICLC) on Feb. 23, 2007 with Accession Number PD 07001 produced an IgMk antibody, showing the same double IgG-Fc₃₄₅₋₃₅₅ and NS3 reactivity as IgM from patients.

The present invention also provides variants of the monoclonal antibody, which are equally or more effective for the scopes of the invention.

For example, derivatives of the antibody endowed with enhanced immunogenicity can be obtained with conventional techniques, for example by addition of an amino acid (C amide) in the COOH region (Stennicke, H R, Biochemistry 1996, 35, 7131-41).

The present invention comprises also antigen binding fragments of the above antibody and recombinant protein mimcking the reactive region of the above antibody (scFv), said antigen binding fragment being capable of the same recognition pattern of the above antibody. Fabs can be obtained by conventional methods, see for example Harlow and lane, Antibodies, A laboratory Manual (1988). For general references in antibody technology, see US2004/0146857 and the references cited therein. For the production of recombinant IgM antibodies, see also Serge E Dolmen, et al, J immunological methods, 2005, 298, 9-20. For the production of scFv, see Kanter G et al, Blood, 2006, 12 Dec. Prepub). This result is important because it reproduces in vivo, the cross-reaction observed in the MC patient and demonstrates, for the first time, a direct cross-reactivity of IgM for the HCV NS3₁₂₃₈₋₁₂₇₉ (immunodominant epitopes) and the IgG-Fc₃₄₅₋₃₅₅ domain. Although the present inventors do not wish to be bound by theoretical considerations, they suggest that the T cell mediated process of clonal selection and somatic mutation, shown in HCV-related lymphoproliferation⁵ and probably induced by HCV-NS3, could lead to the production of RF autoantibodies.

A subsequent increase of RF activity may represent an advantageous phenomenon for some infective agents since it is present in the course of other microbial infections with B-cell expansion (e.g. as a consequence of staphylococcus, HSV-1, HSV-2, varicella-zoster, CMV, and EBV infection) (Oppliger I R et al, A J Exp Med, 1987, 166, 702-710; Nardella F A et al, Scand J Rheumatol Suppl, 1988, 75, 190-198; Stone G C et al, J Immunol, 1989, 143, 565-570).

In further embodiments of the present invention, the above disclosed isolated IgM, the monoclonal antibody and derivatives thereof, the above epitope peptide can be used in methods of diagnosis and treatment of HCV infection and HCV-related diseases.

A further object of the present invention is a method for detecting the presence of the Fc-IgG epitope, said method comprising the step of:

a) contacting a biological sample isolated from a subject infected or suspected to be infected by HCV, with an epitope peptide above disclosed under conditions allowing the formation of an immune complex;

b) determining the formation of said formed complex, wherein the presence of complex formation indicates the detection of specific RF activity.

The methods according to the present invention are carried out with conventional techniques well known in the art. Said methods are disclosed, for example, in US2002/0146685, US 2002/0192639, US 2004/0142321, US 2004/0146857, US 2004/0214994, US 2005/0160142.

The person skilled in the art has the knowledge to understand and carry out the present invention just resorting to the general common knowledge of this technical field. However, for sake of clarity, the following definitions are provided.

The determination of an immune complex is carried out with normal laboratory techniques well known in the art, see for example US 2002/0192639, US 2004/0146857 and references cited therein.

The methods according to the present invention will be carried out by normal means, such as, for example diagnostic kits, which are part of the present invention, comprising the material above disclosed, in particular the isolated IgM, and/or the monoclonal antibody and/or the epitope peptide, together with all reagents and auxiliary material useful for carrying out the method. A package with instructions will form the kit.

The present invention relates also to method for treating an infection, comprising administering to a subject in need thereof the monoclonal antibody and/or its derivatives of the present invention.

In a preferred embodiment, said infection is caused by an infective agent selected from the group consisting of HCV, staphylococcus, HSV-1, HSV-2, varicella-zoster, CMV, and EBV.

In another embodiment, the present invention provides a method for treating HCV-related autoimmune disease comprising administering to a subject in need thereof the monoclonal antibody and/or its derivatives of the present invention. In particular, said disease is type II mixed cryoglobulinemia.

In still another embodiment, the present invention provides a method for treating HCV-related neoplastic disease comprising administering to a subject in need thereof the monoclonal antibody and/or its derivatives of the present invention. In particular, said disease is non-Hodgkin lymphoma with a reactivity against the Fc peptide.

Pharmaceutical compositions useful for the administration of the monoclonal antibody, IgM derivatives and peptide of the present invention are well known in the art and need no particular description. Guidance for making pharmaceutical compositions within the scope of the present invention can be found in Remington's Pharmaceutical Sciences Handbook, last edition, Mack Pub. More specific information can be found in the above cited patents and patents applications, in particular WO 02/022296, US 2004/0146857, US 2004/0214994, and the references cited therein. For the administration of antibodies and peptides, injectable formulations are preferred, but other administration routes can be used. See in particular US 2004/0146857. Formulations in the form of vaccines are preferred.

Monoclonal antibody of the present invention is useful in immunochemistry.

The epitope of Fc-IgG disclosed in the present invention can be used also for selective drug delivery. Once a patient is detected as responder to epitope targeting, a conjugate of the epitope with the drug can be prepared and administered. The conjugate will direct the drug to the expanded B cell, which will be recognized by the epitope and the drug will selectively be released on the cell.

Therefore, another object of the present invention is a method for selecting a patient suffering from an autoimmune or a neoplastic disease related to an infective agent, said agent causing B-cell expansion, said patient to be subjected to treatment for said disease the method comprising:
a)isolating monoclonal IgM from the patient;
b) contacting said monoclonal IgM with the monoclonal antibody of the present invention;
c) determining recognition of said IgM by said antibody;
d) determining epitope region of said IgM;
e) isolating said epitope.

Another object of the present invention is a conjugate of the above isolated epitope with a drug for treating an autoimmune or a neoplastic disease related to an infective agent, said agent causing B-cell expansion.

Another object of the present invention is a method for treating a patient suffering from an autoimmune or a neoplastic disease related to an infective agent, said agent causing B-cell expansion, said patient to be subjected to treatment for said disease the method comprising:
a) selecting the patient with the above method,
b) administering the above conjugate to said selected patient.

The above embodiments of the present invention can be carried out with suitable pharmaceutical compositions containing said conjugate and kits for carrying out the above selection method, said kit and said composition can also be combined in a single package.

Kits for carrying out the above method are commonly used in immunochemistry and immunoassays. Examples of said kits can be found in US 2002/0192639, US 2004/0146857, US 2004/0142321, US 2002/0815774 and related references.

All the references cited are herein incorporated for reference.

The following example further illustrates the invention.

### EXAMPLE

Materials and Methods

Patients

Seventeen HCV-infected and 7 HCV-negative patients with a diagnosis of MC II syndrome, were considered. Sera obtained at diagnosis were tested for anti-HCV antibodies by ELISA (HCV 3.0, Ortho Clinical Systems, Ratitan, N.J., USA) as well as for HCV RNA (Amplicor HCV, Roche Diagnostic Systems, Branchburg, N.Y., USA). All patients were HIV and HbsAg-negative. Four of them had a concomitant B-cell NHL, two, a low grade immunocytoma (patients 1 and 3), one, a nodal diffuse large-cell lymphoma (patient 14), and one a gastric MALT lymphoma (patient 15), the last two without bone marrow involvement. No evidence of any malignant lymphoproliferative disorder was disclosed by physical examination, thorax and abdomen-computed tomography or by bone marrow biopsy in the remaining cases. Seven HCV-negative patients with MC syndrome were selected as negative controls (patients 18-24). MC syndrome was associated with Sjogren's syndrome in three cases (patients 20-22), with chronic hepatitis B virus infection (HBs Ag and HBV DNA-positive in serum) in patient 18 and undifferentiated connective tissue disease in patient 23. A truly “essential” MC syndrome was diagnosed in the remaining patients. Among HCV negative patients only patient 21 also reported a concomitant NHL.

GeneScan Analysis of Clonal B-Cells

PCR reactions for the Ig GeneScanning technique (GS) were performed using previously reported VDJ FR3, specific VH family FR1, and VK fluorescent primers and PCR conditions. Products were subjected to capillary electrophoresis on ABI PRISM 3100. Data were elaborated with 3100 GeneScan 3.7 Software. The peak distribution ranged between 65 and 130 bp for FR3, 310 and 360 bp for FR1, and 120-180, 190-210, 260-300 bp, for VK1/VK6/VK7-JK, VK3-JK, and VK2/VK4/VK7-JK fragments respectively.

Cloning and Sequencing of VH and VK Molecular Clones

VH and VK gene region PCR sequencing was performed as previously reported.

To determine the BCR sequences from oligoclonal B cell pattern, PCR products corresponding to visualized FR1 bands (range from 310 to 360 bp) were cut off from the agarose gel and cloned using the Topo TA Cloning Kit (Invitrogen), as manufactured recommendations. A minimum of twenty randomly selected bacterial colonies was grown. Plasmid DNA was purified (Wizard Plus Miniprep DNA Purification System, Promega) and sequenced using Big Dye Terminators Sequencing Kits as previously described. The most similar VH and VK germline genes were identified by sequence comparison with the International Immunogenetics Database program (http://imgt.cines.fr:/). Deduced amino acid sequences were determined with the Translate BLAST search protein program (http://www.ncbi.nlm.nih.gov/blast/).

Separation and Purification of Cryoprecipitated IgM

Mono/oligoclonal IgM component was purified from cryoprecipitate by gel filtration fractionation on a Hiload Superdex 200 h 26/60 column (Amersham Pharmacia Biotech, Milan, Italy) in acetate buffer pH 4.6, followed by IgM antibody-affinity chromatography (Pharmacia Biotech, Uppsala, Sweden) as previously reported. To remove contaminant IgG we incubated the IgM enriched fractions with protein G sepharose (Pharmacia Biotech, Uppsala, Sweden).

Fingerprinting of IgV Gene Rearrangement and Cryoprecipitated IgM

IgM heavy and light chains were separated by SDS-PAGE on a 12% gel. In-gel trypsin digestion was performed. Matrix Assisted Laser Desorption Ionization Mass Spectrometry (MALDI-TOF) ms was performed using the Voyager-DE PRO Biospectrometry Workstation (Applied Biosystems Inc., Foster City, Calif.) with an accelerating voltage of 20 kV. Each spectrum, was acquired in a mass range between 700 and 4000 Da. Expected monoisotopic tryptic peptide masses of NS3 protein, VH and VL IgM sequences were calculated with the ExPaSy Peptide Mass Tool (http://us.expasy.org/tools). VH/VL peptides shared between BCR and IgM, were searched matching the masses of peptides, theoretically determined by a virtual tryptic digestion of VH and VL sequences from monoclonal B cell DNA and masses of peptides, obtained after the tryptic digestion of heavy and light IgM chains. In the presence of an oligoclonal B cell pattern (exemplary samples: pt 8, 10 and 13), VH/VL sequences were obtained using DNA of twenty random picked bacterial colonies.

Binding of HCV Antigens

As a first approach, INNO-LIA HCV Ab III update ImmunoAssay (LIA, Innogenetics s.r.l., Gent, Belgium) was used with 1 μg of purified IgM. An anti-human antibody specific for IgM isotype and phosphatase-labeled was used as secondary (Kirkegaard & Perry Laboratories-Gaithersburg, Md., USA).

For ELISA assays, plates were coated with 200 ng/well of c22-3 (core protein a.a. sequence 1-120); E2 (a.a. 404-660), c33 (NS3 protein a.a. 1192-1457); C100 (NS4 a.a. 1569-1931); and NS5 (a.a. 2054-2995) HCV proteins (GmbH laboratories Reutlingen, Germany). Additionally, NS3 proteins specific for 1a, 2b, and 2c HCV genotypes (ViroGen, Watertown Mass., USA) were used. 1 μg/well of purified IgM was used as primary antibody. The plates were then washed and incubated with a horseradish peroxidase-labeled goat anti-human IgM (Sigma-chemical Laboratories, St. Louis, Mo., USA). As negative control, the assays were performed with IgM purified from patients with cryoglobulinemia but HCV seronegative (patients 18 to 24). Positive controls consisted of mouse monoclonal antibodies specific for HCV antigens (IBT-Immunological and Biochemical Testsystems GmbH-Germany, and Novocastra, Benton lane, Newcastle upon Tyne UK).

Immunoblotting

Five hundred nanograms of NS3 protein were subjected to 12% SDS-PAGE and transferred to Protran Nitrocellulose Transfer membrane (Schleicher-Schuell, Dassel-Germany). Single lanes were subsequently incubated with 1 μg of purified IgM, from single patients as primary, and with anti-human IgM peroxidase-labeled as secondary antibodies (Sigma chemical Laboratories-St. Louis, Mo., USA). Bands were revealed with a chemiluminescent substrate (ECL Western blot detection reagent, Amersham, API, Indianapolis, Ind.) followed by autoradiography (Hyperfilm ECL Amersham API, Indianapolis, Ind.). IgM purified from patient 20 and an anti NS3 antibody (Novocastra, Benton lane, Newcastle upon Tyne, UK) were used as controls.

Rheumatoid Factor Activity

RF activity was measured by ELISA assay (Sanquin, Central laboratory of the Netherlands Red Cross, Amsterdam, Netherlands) following the manufacturer's instructions but using 1 μg of IgM, instead of patient's serum, as primary antibody. RF activity was calculated in UI/μg of IgM as >200, >100, >12, and <12 UI.

It is known that RF activity is mainly directed against the F_{C} region of IgG in patients with rheumatoid arthritis, however, the target is unknown in case of MC. To test IgM affinity towards the F_{C} portion of IgG, an ELISA assay was performed using purified human IgG F_{C} (MP Biomedical, Irvine, Calif., USA) as coating antigen. The primary antibody consisted of cryoprecipitatecl IgM, while the secondary consisted of an anti human IgM HRP-conjugate. As positive control, we used as primary an anti-human IgG F_{C} specific HRP-conjugate antibody (Sigma-chemical Laboratories, St. Louis, Mo., USA).

To confirm the recognition of epitope F_{C 345-355}, 200 ng of synthetic peptide (IgTech, Paestum, Salento, Italy) were tested in ELISA as described above.

Competitive RF-NS3 Elisa Test

By ELISA, in two exemplary oligo/polyclonal IgM samples which showed a IgG-NS3 cross-reactivity (patients 5 and 7), and in B3-18 hybridoma, NS3 antigen was used as a competitive inhibitor of IgM RF binding. Binding to the IgG-Fc in the presence of excess of fluid phase concentrations of NS3 was expressed as the percent binding of IgM to the same IgG in the absence of NS3 antigen. Serial dilutions, 0 to 20 nmoles of NS3 were incubated with the IgM at concentrations optimal for IgG-Fc binding. The mixtures were then added to IgG-Fc coated wells. Wells were washed, and the amount of RF bound quantified. The same competitive ELISA assay was performed using NS3 protein as coating antigen and IgG-F_{C} as inhibitor.

Epitope Excision Approach

Epitope excision experiment was performed as described. Briefly 0.2 g of CnBr-activated 4B sepharose beads (Amersham Pharmacia Biotech, Milan, Italy) were suspended in 1 mM HCl and pipetted into reaction columns (Handee Mini-Spin Columns, Pierce Biotechnology, Rockford, USA). Thirty μg of purified IgM were added to the beads and incubated for 2 h at room temperature. The beads, were washed with 0.1M Tris-HCl (pH 8), then, equilibrated in PBS and incubated for 2 h at room temperature with antigen (sample) or without antigen (blank). Antigens were HCV NS3 1b (Immunological and Biochemical Testsystems GmbH laboratories), HCV NS3 2b/2c (ViroGen, Watertown Mass., USA), or human IgG F_{C} (ICN Pharmaceuticals, Aurora, Ohio, USA). Trypsin digestion was carried out overnight at 37° C. in 50 mM NH₄HCO₃ buffer (pH 7.8) with an enzyme: substrate ratio of 1:20. Following proteolysis, the beads were washed with digestion buffer, the eluate concentrated under vacuum and then subjected to Zip Tip cleanup (Millipore s.p.a., Milano, Italy) before MALDI-TOF analysis. Affinity bound peptides were dissociated from IgM by addition of 0.1% trifluoroacetic acid (TFA) and the eluate subjected to MALDI-TOF analysis.

B-3 Hybridoma Production

Fifty μg of NS3 peptide GYKVLVLNPSVAAT C(amide) were used to immunize a BALB/c mouse. Mouse spleen cells were fused with NS1 myeloma cells. The cells were then diluted in 20% calf serum DMEM supplemented with HAT medium (Sigma-chemical Laboratories, St. Louis, Mo., USA), and then seeded into microtiter plates. After 3 weeks, ELISA, using NS3 as coating antigen, was employed to test supernatants. Isotype determination of the positive clone (called B-3) was performed with Mouse Typer Sub-Isotyping kit (Bio-Rad, Milano, Italy). B-3 was purified using a Hi Trap IgM purification column. The hybridoma was deposited with Centro di Biotecnologie Avanzate (CBA) Interlab Cell Line Collection (ICLC) on Feb. 23, 2007 with Accession Number PD 07001.

Heavy and light chains were sequenced

B-3 Light Chain IGKV6-15*01/IGKJ2*01

```
Nucleotide sequence
        ACCCAGTCTCCAAAATTCATGTCCACATCAGTAGGAGACAGGGT
CAGCGTCACCTGCAAGGCCAGTCAGAATGTGGGTACTAATGTAGCCTG
GTATCAACAGAAACCAGGGCAATCTCCTAAAGCACTGATTTACTCGGC
ATCCTACCGGTACAGTGGAGTCCCTGATCGCTTCACAGGCAGTGGATC
TGGGACAGATTTCACTCTCACCATCAGCAATGTGCAGTCTGAAGACTT
GGCAGAGTATTTCTGTCAGCAATATAACAGCTATCCTCCAACGTTCGG
AGGGGGCACCAAGCTGGAAATCAAACG
Aminoacid sequence
            T Q S P K F M S T S V G D R V S V T C K A S
Q N V G T N V A W Y
Q Q K P G Q S P K A L I Y S A S Y R Y S G V P D R
F T G S G S G T D F T L
T I S N V Q S E D L A E Y F C Q Q Y N S Y P P T F
G G G T K L E I K
```

B-3 Heavy Chain IGHV9S6*01/J4*01

```
Nucleotide sequence
        ATcCAGTTGGTGCAGTCTGGACCTGAGCTGAAGAAGCCTGGAGA
GACAGTCAAGATCTCCTGCAAGGCTTCTGGGTATACCTTCACAAACTA
TGGAATGAACTGGGTGAAGCAGGCTCCAGGAAAGGGTTTAAAGTGGA
TGGGCTGGATAAACACCAACACTGGAGAGCCAACATATGCTGAAGAG
TTCAAGGGACGGTTTGCCTTCTCTTTGGAAACCTCTGCCAGCACTGCCT
ATTTGCAGATCAACAACCTCAAAAATGAGGACACGGCTACATATTTCT
GTGCAAGATTGAAGCGGTATTTNTATGCTATGGACTACTGGGGTCAAG
GAACC
Aminoacid sequence
    I Q L V Q S G P E L K K P G E T V K I S C K A S
G Y T F T N Y G M
N W V K Q A P G K G L K W M G W I N T N T G E P T
Y A E E F K G R F A
F S L E T S A S T A Y L Q I N N L K N E D T A T Y
F C A R L K R Y X Y A
M D Y W G Q G T
```

B3-18 Hybridoma Production

Fifty μg of NS3 peptide VLVLNPSVAATLGFGAYMSK (IgTecH, Paestum, (SA), Italy) were used to immunize a BALB/c mouse. Mouse spleen cells were fused with NS1 myeloma cells. The cells were then diluted in 20% calf serum DMEM supplemented with HAT medium (Sigma-chemical Laboratories, St. Louis, Mo., USA), and then seeded into microtiter plates. After 3 weeks, ELISA, using NS3 as coating antigen, was employed to test supernatants. Isotype determination of the positive clone (called B3-18) was performed with Mouse Typer Sub-Isotyping kit (Bio-Rad, Milano, Italy). B3-18 was purified using a Hi Trap IgM purification column.

Statistical Methods

Significant tests for qualitative parameters were computed by Fisher's exact test. As the data were not normally distributed non-parametric statistic tests were used. The Wilcoxon rank-test was used to compare the distribution of cryocrit percentage and serum RF concentration between two groups of patients (i.e., NS3-positive IgM and NS3 negative IgM patients). Results were considered to be statistically significant when p<0.05.

Results

Identity Between BCR of Monoclonal B Cells and Cryoprecipitated IgM

To begin searching for a BCR ligand potentially involved in inducing and maintaining clonal B cell proliferation, two experiments were performed in parallel. First, IgH VDJ gene rearrangement from bone marrow specimens of patients with type II MC was examined by PCR. A polyclonal pattern was observed in 7 subjects (samples 7, 9, 11, 14-17), an oligoclonal pattern in 7 subjects (samples 4, 5, 6, 8, 10, 12, 13) and a frank monoclonal pattern in 3 subjects (sample 1-3). Nucleotide VH and VL sequences were deposited in the NCBI GenBank (AF301518, AY703067, AY703068, AY704914, AY703069, AY703066). In the second experiment, purified IgM from serum cryoprecipitates were analyzed by MALDI-TOF mass spectrometry. In the 3 patients with a B-cell monoclonality, the peptide mass fingerprinting of IgM matched the fingerprinting of BCR sequences as generated by the Peptide Mass Expasy Tool—

TABLE 1

Comparison of theoretically calculated peptide masses of clonal
B-cell and experimental peptide masses from the in-gel digestion of
the VH and VK cryoprecipitate IgM. Only specific patient's Ig-V
peptides are reported, mutations with respect the most similar
germline gene are evidenced in bold face.

| | | | | |
|---|---|---|---|---|
| | | | Theoretically | |
| | | Peptides from variable | calculated | Measured |
| Patient | Residues | chain region | mass (Da) | mass (Da) |

| | | | | |
|---|---|---|---|---|
| 1 | | Heavy Chain V1-2/D2-15/J4 | | |
| | germline | ASGYTFTGYYMHWVR | 1838.83 | |
| | 24-38 patient | ASGYTFTDYYIHWVR | 1879.05 | 1879.12 |
| | germline | VTMTRDTSISTVYMELSR | 2090.02 | |
| | 68-85 patient | VTVTRDTSINTVYMELSR | 2085.06 | 2085.26 |
| | | Light Chain V3-15/J1 | | |
| | germline | ASQSVSSNLAWYQQKPGQAPR | 2303.1527 | |
| | 25-54 patient | ASQSVSSNLAWYQQQPGQAPR | 2302.12 | 2301.84 |
| 2 | | Heavy Chain V1-69/D3-22/J4 | | |
| | germline | ASGGTFSSYAISWVR | 1588.7754 | |
| | 43-57 patient | ASGGTFSSYGISWVR | 1574.7603 | 1574.81 |
| | germline | STSTAYMELSSLR | 1445.6941 | |
| | 94-106 patient | STSTVYMELTSLR | 1487.7416 | 1487.76 |
| | | Light Chain V3-20/J1 | | |
| | germline | LLIYGASSR | 979.5571 | |
| | 29-37 patient | LLVYGASNR | 992.55 | 992.32 |
| | germline | ATGIPDRFS | 963.4894 | |
| | 38-46 patient | ATGIPDRFR | 1032.56 | 1032.42 |
| | germline | GSGSGTDFTLTISR | 1398.6859 | |
| | 47-60 patient | GSGSGTDFTLTITR | 1412.70 | 1412.53 |
| | germline | LEPEDFAVYYCQQYGSSPR | 2251.9964 | |
| | 61-79 patient | LEPEDFALYYCQQYGSSPR | 2323.0341 | 2322.85 |
| | germilne | SQSVSSSYLAWYQQKPGQAPR | 2368.1680 | |
| | 7-28 patient | TSQSVSSTYLAWYQQTPGQAPR | 2456.1846 | 2455.94 |
| 3 | | Heavy Chain V3-7/D3-22/J4 | | |
| | germline | QDGESEKYYVDSVK | 1646.7544 | |
| | 61-75 patient | EDGESEKYYVDSVK | 1518.69 | 1518.81 |
| | germline | NSLYLQMNSLR | 1338.6834 | |
| | 83-87 patient | NSLDLQMSSLR | 1263.63 | 1263.72 |
| | | light chain V3-15/J1 | | |
| | germline | ASQSVSSNLAWYQQKPGQAPR | 2303.1527 | |
| | 25-46 patient | ASQSVSSNLAWYQQRPGQAPR | 2331.16 | 2331.46 |
| 8 | | Heavy Chain VH4-59 | | |
| | germline | VTISIDVSK | 961.5564 | |
| | 66-74 patient | VTISIDTSK | 963.53 | 963.61 |

Identify by maldi-tof mass spectrometry VK peptides from patient 8 and both VH and VK peptides from patient 10 and 13 are unmutated with respect the most similar germline genes. Thus, they are not reported.

Moreover, peptides attributed to both the patient variable heavy and light chains carry several replacement mutations respect to the most similar germline genes and this result corroborates the identity between variable region of BCR and that of cryoprecipitate IgM. In 3 HCV-infected patients tested by fingerprinting and displaying a restricted oligoclonal-B-cell pattern (sample 8, 10 and 13), although intraclonal diversity may be present and IgM is often oligoclonal, some peptides shared between some BCRs and cryoprecipitated IgMs have been found. Table 1 reports a specific peptide, founded in sample 8, which showed a mutation shared between the BCR and the IgM. Other peptides were identical to germline; thus, they were not reported. Comparison of BCR/IgM fingerprinting from polyclonal B cell samples were not performed due to the too high number of B cell clones that should be analyzed to obtain an adequate information. In these cases, it is possible that several bone marrow B cell clones contribute to the oligo/polyclonal production of these IgMs, and/or, that antibody secreting cells (ASC) responsible of the cryoprecipitable IgM component are confined in a compartment different from bone marrow. This concept is supported by the demonstration that, under chronic inflammatory conditions, B cells bearing antigen-specific receptors can be stimulated to proliferate and differentiate into ASC within ectopic sites, including the germinal centers of intraportal lymphoid liver follicles in HCV infection. Nevertheless, by analogy to that we shown for the bone marrow with monoclonal B-cell, cryoprecipitate IgMs should represent the counterpart of ectopic ASCs.

Monoclonal B-Cell of Chronically HCV-Infected Patients Bind HCV NS3 Protein

By LIA test, cryoprecipitated IgM from 2 out of the 3 patients with a monoclonal B cell pattern and 4/14 HCV-positive patients with an oligo-polyclonal pattern showed a weak reactivity against HCV-NS3 protein. ELISA and Western Blot were used to confirm reactivity against NS3. The E2, core, NS4A, NS4B and NS5 HCV antigens were not bound by the same IgM. Furthermore, there was no signal visible when IgM purified from 7 selected HCV-negative patients with type II MC were used. Since NS3 protein immobilized in the LIA strip is specific for the 1b HCV-genotype, an additional ELISA assay was carried out with recombinant NS3 protein specific for the patient's HCV genotype. Two additional patients (patients 3 and 15), were positive with HCV-NS3 2b and 2c genotype, respectively.

NS3 reactivity is related to higher production of cryoglobulins (p=0.01 Wilcoxon test) but not to the value of RF (n.s. Wilcoxon test).

Monoclonal B-Cell of Chronically HCV-Infected Patients Shows RF Activity

Since IgM are often polyreactive and cryoprecipitated IgM usually have RF activity, we verified whether IgM showed reactivity against human IgG. All samples showed RF activity by standard ELISA. RF activities positively correlate with serum RF concentrations, Fisher exact test p=0.03.

Monoclonal IgM of Chronically HCV-Infected Patients Recognize Distinct Epitopes

To verify if the cross reactivity against NS3 and IgG was due to mimicry between these two antigens, we attempt to define the NS3 and IgG epitopes recognized by IgM. The epitope analysis was performed only for patients 1, 3, 5 and 7 from whom it was possible to obtain sufficient IgM for the analysis. All of these IgM recognized the Fc portion of IgG in ELISA. They were therefore incubated with either polyclonal human-IgG Fc fragment or genotype-specific HCV-NS3 protein.

Results for NS3 show 3 different sets of affinity bound peptides: 2038.2 Da (for patient 1), 991.5 Da (for patient 3) and 1138.6, 1152.6, 1589.8 Da (for both patients 5 and 7). Fragment mass from patient 1 (who has HCV 1b genotype and a monoclonal B-cell proliferation) corresponds to peptide 1251-1270: VLVLNPSVAATLGFGAYMSK; for patient 3 (who has a HCV 2b genotype and a monoclonal proliferation) the mass corresponds to peptide 1271-1279: AHGINPNIR; and, for both patients 5 and 7 (who have HCV 1b genotype and an oligoclonal/polyclonal B-cell pattern), the masses correspond to multiple peptides: 1238-1248: VPAAYAAQGYK; 1284-1298: TITTGSPITYSTYGK and 1379-1387:AIPLEVIK (Table 2).

TABLE 2

| | | |
|---|---|---|
| Patient 1 | Patient 3 | Patient 5-7 |

| | | |
|---|---|---|
| VLVLNPSVAATLGFGAYMSK ₁₂₅₁₋₁₂₇₀ | AHGINPNIR ₁₂₇₁₋₁₂₇₉ | VPAAYAAQGYK ₁₂₃₈₋₁₂₄₈ |
| | | TITTGSPITYSTYGK ₁₂₈₄₋₁₂₉₈ |
| | | AIPLEVIK ₁₃₇₉₋₁₃₈₇ |

Interestingly, IgM bound peptides are adjoining segments localized in the helicase domain of the NS3 protein in a region exposed to the solvent. In contrast, for the IgG-Fc, the mass of the affinity bound peptide (1286.7 Da) is unique for all the samples tested. This mass matched (Mascot Data Base) the monisotopic mass of the F_{C345-355} EPQVYTLPPSR sequence of the CH3 region of human IgG₁, IgG₂ and IgG₃. Moreover, we confirmed by ELISA that the synthetic peptide, Fc₃₄₅₋₃₅₅, reacted with 13/17 IgM tested.

B3-18 Hybridoma Recognized Fc₃₄₅₋₃₅₅ Peptide

To verify the hypothesis that an anti-NS3 IgM would be reactive against an IgG-F_{C} antigen, we used the synthetic peptide corresponding to the NS3 epitope recognized by IgM from patient 1, to immunize a BALB-c. A positive ELISA clone (B3-18) was selected, expanded and resulted in an IgMk antibody. This antibody, as ascertained by epitope excision and ELISA, identified the same F_{C345-355} EPQVYTLPPSR peptide, and thus possesses the same dual reactivity as IgM from patient 1.

NS3 Inhibition of IgM-RF Binding to IgG

Competitive RF-NS3 Elisa test demonstrated that NS3 partially inhibited the IgM binding of IgG-Fc in both the exemplary oligo/polyclonal samples tested (samples 5 and 7), as well as in the B3-18 hybridoma. Moreover, IgG-Fc binding inhibition was achieved by preincubation with lower NS3 concentrations for B3-18 than for sample 5 and 7. IgG-Fc fragment completely inhibited the IgM binding to NS3 for both samples 5 and 7, while only partially inhibited B3-18.

1. A monoclonal antibody or a derivative thereof which is cross-reactive against the immunogenic sequence of an infective agent causing a B-cell expansion and IgG-Fc.

2. A monoclonal antibody or a derivative thereof according to claim 1, wherein said infective agent is selected from the group consisting of staphylococcus, HCV, HSV-1, HSV-2, varicella-zoster, CMV, and EBV.

3. A monoclonal antibody or a derivative thereof, according to claim 2, which is cross-reactive against helicase domain of HCV-NS3 and human IgG-Fc.

4. Antibody or a derivative thereof according to claim 3, which is cross-reactive against NS3₁₂₄₆₋₁₂₅₈ and IgG-Fc₃₄₅₋₃₅₅.

5. Antibody or a derivative thereof according to claim 3, which is cross-reactive against NS3₁₂₄₆₋₁₂₅₈ and IgG-Fc₃₄₅₋₃₅₅ and has the following light and heavy chain sequences, respectively:
(SEQ ID NO: 8)
TQSPKFMSTSVGDRVSVTCKASQNVGTNVAWYQQKPGQSPKALIYSASYR
YSGVPDRFTGSGSGTDFTLTISNVQSEDLAEYFCQQYNSYPPTFGGTKLE
IK
and
(SEQ ID NO: 10)
IQLVQSGPELKKPGETVKISCKASGYTFTNYGMNWVKQAPGKGLKWMGWI
NTNTGEPTYAEEFKGRFAFSLETSASTAYLQINNLKNEDTATYFCARLKR
YXYAMDYWGQGT.

6. Antibody or a derivative thereof according to claim 3, which is cross-reactive against NS3₁₂₄₆₋₁₂₅₈ and IgG-Fc₃₄₅₋₃₅₅.

7. Hybridoma producing the antibody of claim 6 deposited with Centro di Biotecnologie Avanzate (CBA) Interlab Cell Line Collection (ICLC) on February 2007 with Accession Number PD 07001.

8. Isolated monoclonal IgM from a patient suffering from an infection from an infective agent causing a B-cell expansion.

9. Cryoprocipitable IgM according to claim 8, wherein said patient suffers from with type II mixed cryoglobulinemia.

10. An isolated peptide epitope of IgG-Fc having the sequence EPQVYTLPPSR (SEQ ID NO:3).

11-12. (canceled)

13. Pharmaceutical composition comprising the IgG-Fc peptide epitope of claim 10, in admixture with conventional vehicles and excipients.

14-20. (canceled)

21. An isolated peptide epitope obtained by a method comprising:
a. isolating monoclonal IgM from a patient suffering from type II mixed cryoglobulinemia, said cryoglobulinemia being related to hepatitis C virus (HCV) infection;
b. contacting said monoclonal IgM with a monoclonal antibody produced by the hybridoma deposited with Centro di Biotecnologie Avanzate (CBA) Interlab Cell Line Collection (ICLC) on Feb. 23, 2007 with Accession Number PD 07001;
c. determining recognition of said IgM by said antibody;
d. determining a peptide epitope region of said IgM; and
e. isolating said peptide epitope.

22. (canceled)

23. A conjugate of the IgG-Fc peptide epitope of claim 21 with a drug suitable for treating type II mixed cryoglobulinemia.

24. A pharmaceutical composition comprising the conjugate of claim 23.

25. A method for treating a patient suffering from type II mixed cryoglobulinemia, said patient to be subjected to treatment for said disease the method comprising:
a. selecting the patient, and
b. administering a conjugate of the peptide epitope of claim 10 with a drug suitable for treating the type II mixed cryoglobulinemia.

26 (canceled)

27. Pharmaceutical composition comprising the IgG-Fc peptide epitope of claim 10 and an immunodominant region of the HCV-NS3₁₂₃₈₋₁₂₇₉ region having the sequence VPAAYAAQGYKVLVLNPSVAATLGFGAYMSKAHGIDPNIR (SEQ ID NO:1), in admixture with conventional vehicles and excipients.

28. Pharmaceutical composition comprising the IgG-Fc peptide epitope of claim 10 and an immunodominant region of the HCV-NS3₁₂₃₈₋₁₂₇₉ region having the sequence GYKVLVLNPSVAAT C(amide) (SEQ ID NO:2), in admixture with conventional vehicles and excipients.

29. A conjugate of the IgG-Fc peptide epitope of claim 10 with a drug suitable for treating type II mixed cryoglobulinemia.

30. A pharmaceutical composition comprising the conjugate of claim 23.
